(19) United States
(12) Patent Application Publication
(10) Pub. No.: US 2026/0248376 A1
(43) Pub. Date: Aug. 27, 2026

(54) GASTRO RECTAL OSCOPY DEVICE

(71) Applicants: Ghulam MUJTABA, Las Vegas, NV (US); Ori LEDANY, Las Vegas, NV (US)

(72) Inventors: Ghulam MUJTABA, Las Vegas, NV (US); Ori LEDANY, Las Vegas, NV (US)

(21) Appl. No.: 19/548,226

(22) Filed: Feb. 24, 2026

Related U.S. Application Data
(60) Provisional application No. 63/762,535, filed on Feb. 24, 2025.

(51) Int. Cl. A61B 1/31; A61B 1/00; A61B 1/005; A61B 1/04; A61B 1/06

(52) U.S. Cl. CPC A61B 1/31; A61B 1/00082; A61B 1/00101; A61B 1/0052; A61B 1/042; A61B 1/0638; A61B 1/0692

(57) ABSTRACT

A gastro rectal oscopy system that includes a body portion, a base portion, a vibratory device and an inflatable device. The body portion has a proximal end and a distal end, wherein intermediate the proximal end and the distal end. The body portion has a plurality of elongated openings formed therein. The head portion is attached to the distal end. The head portion has at least one light source mounted therein. The base portion is attached to the proximal end. The base portion has a control provided thereon and a connector. The vibratory device is positioned inside of at least one of the body portion, the head portion and the base portion. The inflatable device is provided inside of the body portion. The inflatable device is positionable in an uninflated configuration and an inflated configuration. When the inflatable device is in the uninflated configuration, the inflatable device does not extend above an outer surface of the body portion. When the inflatable device is in the inflated configuration, the inflatable device extends through the plurality of openings above the outer surface of the body portion. The connector is operably attached to the inflatable device for changing the inflatable device between the uninflated configuration and the inflated configuration.

### REFERENCE TO RELATED APPLICATION

This application claims priority to Provisional Applic. No. 63/762,535, filed on Feb. 24, 2025, the contents of which are incorporated herein by reference.

### FIELD OF THE INVENTION

The invention relates generally to medical devices. More particularly, the invention relates to a gastro rectal oscopy device.

### BACKGROUND OF THE INVENTION

There are a variety of health conditions associated with the human rectum that require medical treatment. Heretofore, there has not been a device that enables efficient visualization and treatment of these health conditions.

### BRIEF DESCRIPTION OF THE DRAWINGS

The accompanying drawings are included to provide a further understanding of embodiments and are incorporated in and constitute a part of this specification. The drawings illustrate embodiments and together with the description serve to explain principles of embodiments. Other embodiments and many of the intended advantages of embodiments will be readily appreciated as they become better understood by reference to the following detailed description. The elements of the drawings are not necessarily to scale relative to each other. Like reference numerals designate corresponding similar parts.

FIG. 1 is a side view of a gastro rectal oscopy device according to an embodiment of the invention.

FIG. 2 is an end view of the device of FIG. 1.

FIG. 3 is a side view of the device of FIG. 1 in a deflated configuration.

FIG. 4 is a side view of the device of FIG. 1 in an inflated configuration.

FIG. 5 is a side view of an alternative embodiment of the gastro rectal oscopy device.

FIG. 6 is a side view of another alternative embodiment of the gastro rectal oscopy device.

FIG. 7 is a side view of a sleeve for use with the gastro rectal oscopy device of Fig.

FIG. 8 is a side view of an adapter.

FIG. 9 is a top view of the adapter of FIG. 8.

### DETAILED DESCRIPTION OF THE INVENTION

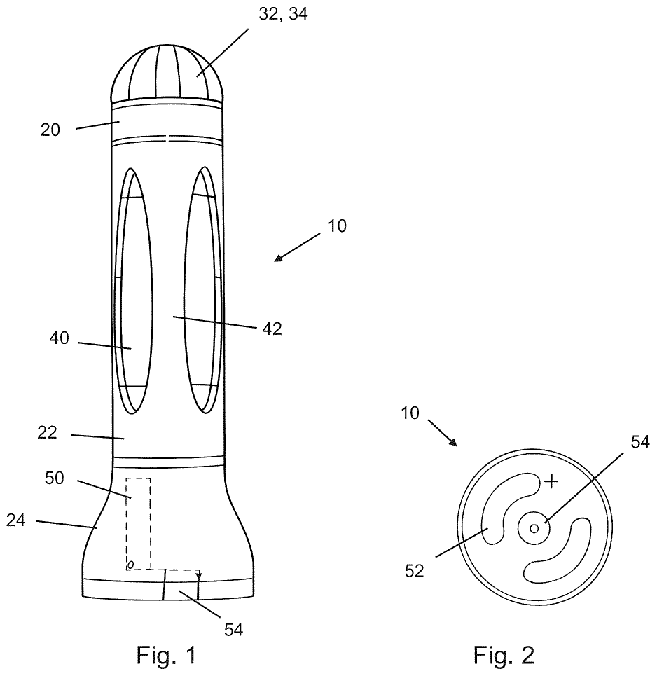

An embodiment of the invention is directed to a gastro rectal oscopy device as illustrated at 10 in FIG. 1. The gastro rectal oscopy device 10 generally includes a head portion 20, a body portion 22 and a base portion 24.

In certain embodiments, at least part of the head portion 20 is fabricated from a transparent material. In one configuration, the head portion 20 includes 8 transparent regions 30.

In certain embodiments, there are two sets of light sources 32, 34 that are capable of illuminating in different colors. In one configuration, first light sources 32 are capable of illuminating in the color red at a wavelength of about 660 nanometers and the second light sources 34 are capable of illuminating in the color blue at a wavelength of about 460 nanometers. The first light sources 32 and the second light sources 34 may be placed in an alternating pattern in the head portion 20. There may be 8 of the first light sources 32 and 8 of the second light sources 34. The 2 different light sources 32, 34 may be used in conjunction with different procedures.

In one configuration, the head portion 20 has a length of about 1 inch and a diameter of about ¾ of an inch. A person of skill in the art will appreciate that the length and the width of the head portion 20 may vary so that the gastro rectal oscopy device 10 is suited for use on different size patients such as adults, adolescence and youth.

The gastro rectal oscopy device 10 includes circuitry (not shown) that controls the illumination of the first and second light sources 32, 34. A person of skill in the art will appreciate that the circuitry may assume a variety of configurations using the concepts of the invention.

The body portion 22 may have a generally cylindrical shape. In certain embodiments, a shape and a size of an outer surface of the body portion 22 is substantially the same as a shape and a size of an outer surface of the head portion 20.

Intermediate opposite ends thereof, the body portion 22 includes a plurality of elongated openings 40 formed therein. In one such embodiment, there are 4 of the elongated openings 40. While it is illustrated that the elongated openings 40 are spaced at similar distances between each other, it is possible to use other configurations.

The elongated openings 40 may have a generally oval shape. While it is illustrated at the elongated openings 40 each have a similar shape, it is possible for other configurations to be used.

Each of the elongated openings 40 has a length that is at least 5 times greater than a width of the openings 40. In one such embodiments, the length of the elongated openings 40 is about 10 times the width of the elongated openings 40.

Each of the elongated openings 40 extends more than 50 percent of the length of the body portion 22. In certain embodiments, the length of the elongated openings 40 is about 80 percent of the length of the body portion 20.

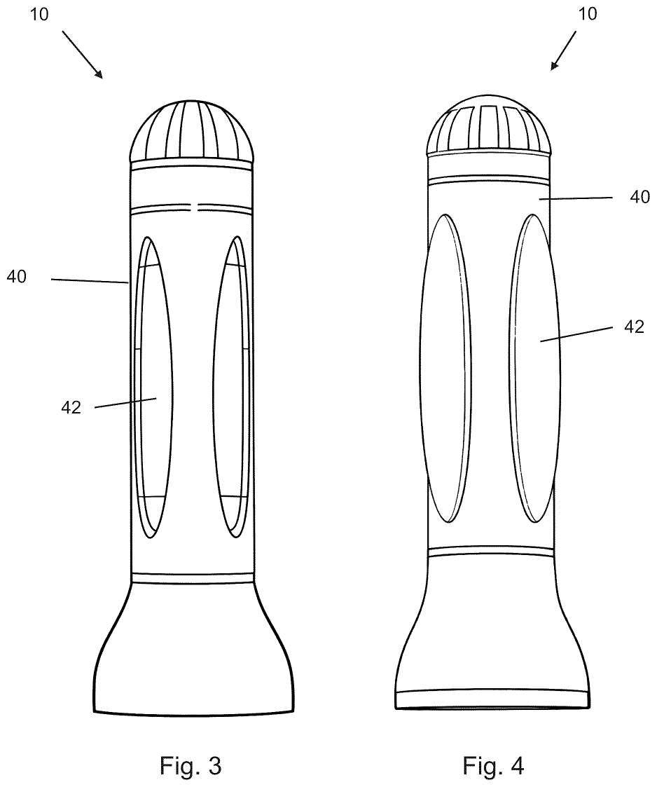

An inflatable device 42 is positioned inside of the body portion 22. When the inflatable device 42 is in an uninflated configuration (illustrated in FIG. 3), the inflatable device 42 does not extend above a surface of the body portion 22. When the inflatable device 42 is in an inflated configuration (illustrated in FIG. 4), the inflatable device 42 extends above the surface of the body portion 22.

A variety of techniques may be used to move the inflatable device 42 between the uninflated configuration and the inflated configuration. An example of one suitable technique for inflating the inflatable device 42 is air.

Air may be provided by a pneumatic system (not shown) that is separate from the gastro rectal oscopy device 10. In certain embodiments, the pneumatic system is connected to the inflatable device 42 using an elongated tubing.

A variety of techniques may be used for removably attaching the body portion 22 to the head portion 20. Examples of two such suitable techniques are a half twist locking mechanism and a threaded mechanism. In certain embodiments, the body portion 22 has a length of about 2 ½ inches.

The base portion 24 may be outwardly flared proximate a lower end thereof to limit a distance that the gastro rectal oscopy device 10 may be inserted into the rectum. An end of the base portion 24 that is adjacent to the body portion 22 may have a shape and a size that is substantially the same as the shape and the size of the body portion 22.

Mounted inside of at least one of the base portion 24, the body portion 22 and the head portion 20 is a vibratory device 50 that is capable of providing sonic vibrations during the use of the gastro rectal oscopy device 10. One suitable power source for the vibratory device 50 is at least one battery (not shown) that is mounted inside of the gastro rectal oscopy device 10.

A control 52 is provided on a lower surface of the base portion 24, as illustrated in FIG. 2. The control 52 enables changing the vibration intensity of the vibratory device 50. A person of skill in the art will appreciate that the control 50 may assume a variety of configurations using the concepts of the invention.

The base portion 24 also includes a connector 54 for attaching tubing that is capable of delivering air to inflate the inflatable device 42.

A variety of techniques may be used for removably attaching the base portion 24 to the body portion 22. Examples of two such suitable techniques are a half twist locking mechanism and a threaded mechanism. In certain embodiments, the base portion 24 has a length of about 1 ⅛ inches.

When the inflatable device 42 is in the inflated configuration, the portions of the inflatable device 42 that extend through the elongated openings 40 as illustrated in FIG. 4 such that the inflatable device 42 is in contact with an inner wall of the rectum.

This contact between the inflatable device 42 and the inner wall of the rectum can be used to transfer sonic waves to the rectal nerves and muscles. This process also causes an increase in blood flow and helps with pain reduction and enhances recovery times.

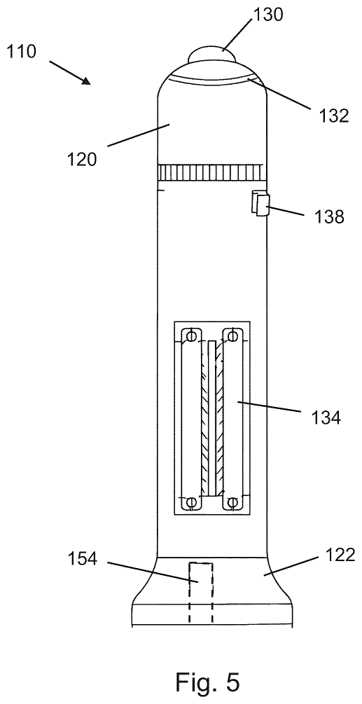

Another component of the invention is a diagnostic transducer, which is illustrated at 110 in FIG. 5. The diagnostic transducer 110 includes a head portion 120 and a body portion 122.

The head portion 120 may has a generally rounded shape at a distal end thereof. A camera 130 is provided in the head portion 120. In certain embodiments, the camera 130 is at the distal end of the head portion 120.

The head portion 120 also includes a lighting mechanism 132 proximate the camera 130 to enhance the ability to capture images using the camera 130. In certain embodiments, the lighting mechanism 132 has a generally circular shape and extends substantially around the camera 130.

The diagnostic transducer 110 includes a temperature sensor 124. In certain embodiments, the temperature sensor 124 is located proximate the head portion 120. A person of skill in the art will appreciate that the temperature sensor 124 may assume a variety of configurations using the concepts of the invention.

The body portion 122 may have a generally cylindrical shape. In certain embodiments, a shape and a size of an outer surface of the body portion 122 is substantially the same as a shape and a size of an outer surface of the head portion 120.

Proximate a distal end of the body portion 122, the body portion 122 has an air valve 138 for injecting air into the rectum similar to what is done when performing a colonoscopy.

Mounted inside of the body portion 122 is an ultrasonic wave emitting mechanism 134 that is capable of delivering ultrasonic waves to the inner wall of the rectum after the device is inserted into the rectum. A person of skill in the art will appreciate that the ultrasonic wave emitting mechanism 134 may assume a variety of configurations using the concepts of the invention. An ultrasonic wave intensity control 136 may be provided on the ultrasonic wave emitting mechanism 134.

A proximal end of the body portion 122 includes a connector 154 for attaching an air tube for delivering air to the valve 130. A person of skill in the art will appreciate that the connector 154 may assume a variety of configurations using the concepts of the invention.

A variety of techniques may be used for removably attaching the body portion 122 to the head portion 120. Examples of two such suitable techniques are a half twist locking mechanism and a threaded mechanism.

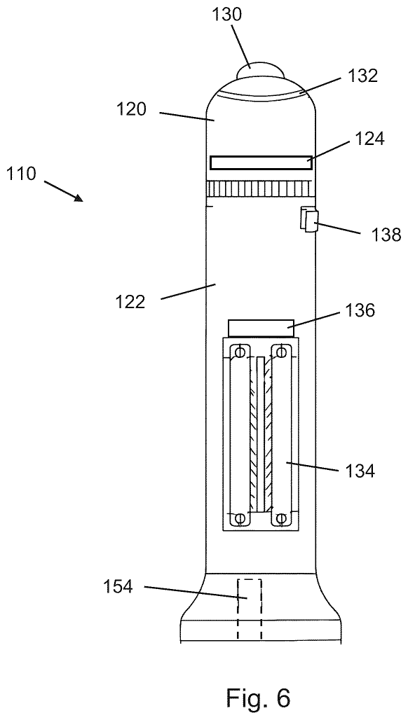

An alternative embodiment of the diagnostic transducer 110 is illustrated in FIG. 6. The features and configuration of this embodiment of the diagnostic transducer 110 are similar to the diagnostic transducer 110 illustrated in FIG. 5. However, this embodiment of the diagnostic transducer 110 includes a temperature sensor 124 proximate a distal end of the diagnostic transducer 110 to facilitate obtaining temperature data from inside the rectum while using the diagnostic transducer 110.

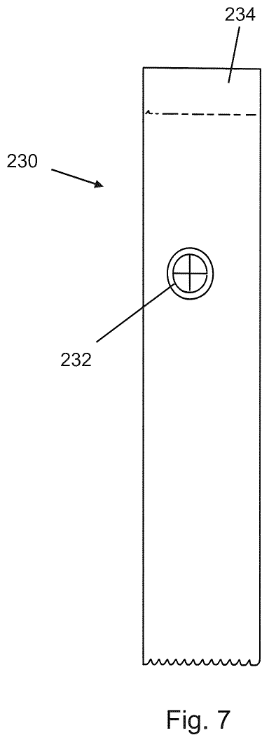

A sleeve 230 such as illustrated in FIG. 7 may be positioned at least partially over the diagnostic transducer 110 during use. The sleeve 230 may enhance the ability to reuse the diagnostic transducer 110 in a sterile manner.

The sleeve 230 may have a length and a width that are similar to a length and a width of the diagnostic transducer 110 such that the portion of the diagnostic transducer 110 that is extended into the rectum is covered by the sleeve 230.

The sleeve 230 may include an opening 232 that is positioned on the sleeve 230 to correspond with the location of the air valve 130 on the diagnostic transducer 110. The opening 232 may be formed with a shape and a size that are similar to a shape and a size of the air valve 130.

The sleeve 230 may be fabricated from a variety of materials using the concepts of the invention. Examples of 2 suitable materials that may be used to fabricate the sleeve 230 are plastic and latex. If the sleeve 230 is fabricated from latex, a distal end 234 of the sleeve 230 may be fabricated from a more durable material such as plastic to reduce the potential of an opening forming in the sleeve 230 as the diagnostic transducer 110 is inserted into the rectum.

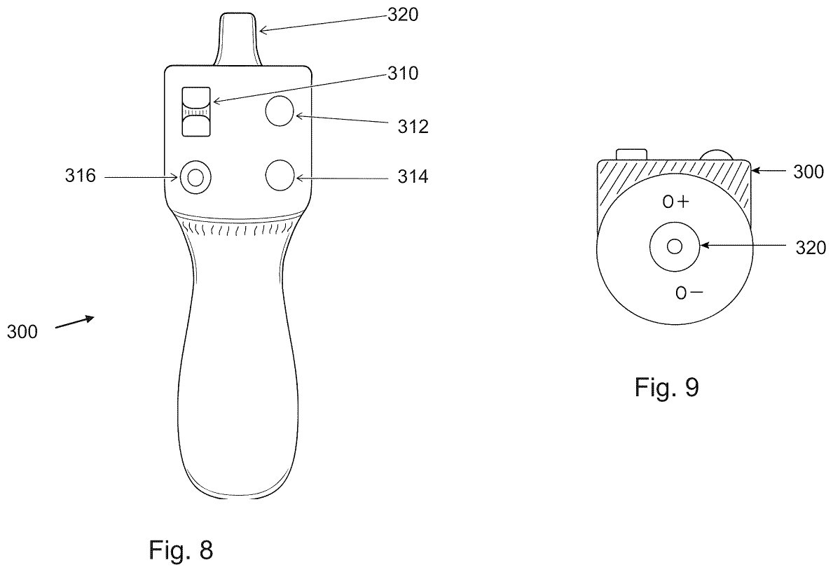

An adapter handle 300, which is illustrated in FIG. 8, may be attached to a proximal end of the diagnostic transducer 110. In certain embodiments, the diagnostic transducer 110 is removably attached to the adapter handle 300. Using such a configuration enables different diagnostic transducers 110 to be used with the adapter handle 300.

The adapter handle 300 may be formed with a generally cylindrical shape to facilitate gripping by a person's hand. To further enhance the ability of the person to grip the adapter handle 300, a diameter of the adapter handle 300 near the distal end thereof may be smaller than a diameter of the adapter handle 300 proximate a proximal end thereof.

Wires to power the operation of the components in the diagnostic transducer 110 extend through the adapter handle 300. A variety of mechanisms may be used to connect the wires 330 to the contacts on the proximal end of the diagnostic transducer 110. One suitable technique is a spring contact.

Similarly, the tubing for air extends through the adapter handle 300. A variety of mechanisms may be used to engage the tubing to the tubing in the diagnostic transducer 110. An example of one suitable technique is a pressure fit.

The distal end of the adapter handle 300 is configured to mate with the proximal end of the diagnostic transducer 110. A person of skill in the art will appreciate that a variety of techniques may be used for the adapter handle 300 to detachably engage the diagnostic transducer 110. An example of one suitable mechanism for detachably engaging the adapter handle 300 to the diagnostic transducer 110 is a half twist lock mechanism.

The adapter handle 300 may include an air flow control 310, a light control 312, a sonic wave control 314 and a camera control 316. A distal end of the adapter handle 300 may include an air connector 320. In certain embodiments, the air connector 320 utilizes a friction fit mechanism.

A control unit (not shown) is operably attached to the adapter handle 300. The control unit includes a plurality of controls that enable adjustment of aspects such as the air flow rate. The control unit may also include a display that enables viewing of images captured by the camera 130.

Both of the devices are anticipated to be used on a patient during a single visit. The first device is designed for treatments while the second device is designed for diagnostics and treatments.

The first device is particularly suited for procedures such as anorectal biofeedback aftercare/manometry, exercise the rectal walls and pelvic floor muscles, manipulate the nerves towards normal response, reduce pain, improve sphincter tone with sonic waves and LED lights, speeding recovery for anal fissure and hemorrhoids.

The second device is particularly suited for procedures such as receive the inner rectal vision for general diagnostics (like a mini colonoscopy but limited to the anorectal only), exercise the rectal walls and pelvic floor muscles, manipulate the rectal nerves towards normal response, detect swelling in the prostate gland and reduce swelling and promote normal urinary function by using the LED lights and the ultrasonic waves mechanism and speeding recovery for anal fissure and hemorrhoids.

The devices of the invention thereby fulfill the need for new therapies for treating anorectal problems. In many situations, it is possible to use the devices without the patient receiving anesthesia.

In the preceding detailed description, reference is made to the accompanying drawings, which form a part hereof, and in which is shown by way of illustration specific embodiments in which the invention may be practiced. In this regard, directional terminology, such as “top,” “bottom,” “front,” “back,” “leading,” “trailing,” etc., is used with reference to the orientation of the Figure(s) being described. Because components of embodiments can be positioned in a number of different orientations, the directional terminology is used for purposes of illustration and is in no way limiting. It is to be understood that other embodiments may be utilized and structural or logical changes may be made without departing from the scope of the present invention. The preceding detailed description, therefore, is not to be taken in a limiting sense, and the scope of the present invention is defined by the appended claims.

It is contemplated that features disclosed in this application, as well as those described in the above applications incorporated by reference, can be mixed and matched to suit particular circumstances. Various other modifications and changes will be apparent to those of ordinary skill.

1. A gastro rectal oscopy system comprising:
a body portion having a proximal end and a distal end, wherein intermediate the proximal end and the distal end, the body portion has a plurality of elongated openings formed therein;
a head portion attached to the distal end, wherein the head portion has at least one light source mounted therein, a base portion attached to the proximal end, wherein the base portion has a control provided thereon and a connector;
a vibratory device positioned inside of at least one of the body portion, the head portion and the base portion; and
an inflatable device provided inside of the body portion, wherein the inflatable device is positionable in an uninflated configuration and an inflated configuration, wherein when the inflatable device is in the uninflated configuration, the inflatable device does not extend above an outer surface of the body portion, wherein when the inflatable device is in the inflated configuration, the inflatable device extends through the plurality of openings above the outer surface of the body portion and wherein the connector is operably attached to the inflatable device for changing the inflatable device between the uninflated configuration and the inflated configuration.

2. The gastro rectal oscopy system of claim 1, wherein the body portion has a generally cylindrical shape, wherein the elongated openings are provided around a periphery of the body portion and wherein the base portion is outwardly flared proximate an end thereof that is opposite the body portion.

3. The gastro rectal oscopy system of claim 1, wherein each of the elongated openings has a length and a width, wherein the elongated opening length is between about 5 times and about 10 times greater than the elongated opening width and wherein the elongated opening length is between about 50 percent and about 80 percent of a length of the body portion.

4. The gastro rectal oscopy system of claim 1, wherein the at least one light source comprises a first light source and a second light source, wherein the first light source is capable of emitting light at a first wavelength, wherein the second light source is capable of emitting light at a second wavelength and wherein the first wavelength is different that the second wavelength.

5. The gastro rectal oscopy system of claim 1, wherein the head portion detachably engages the body portion and wherein the based portion detachably engages the body portion.

6. The gastro rectal oscopy system of claim 1, and further comprising an adapter handle that is capable of engaging the base portion, wherein the adapter handle includes at least one control for controlling the operation of the gastro rectal oscopy system.

7. The gastro rectal oscopy system of claim 6, wherein the at least one control comprises an air flow control, a light control, a sonic wave control and a camera control.

8. A gastro rectal diagnostic transducer system comprising:
a body portion having a proximal end and a distal end, wherein intermediate the proximal end and the distal end;
a head portion attached to the distal end, wherein the head portion has a camera and at least one light source mounted therein, a base portion attached to the proximal end;
ultrasonic wave emitting mechanism positioned inside of at least one of the body portion, the head portion and the base portion; and
an air valve providing in at least one of the body portion and the head portion.

9. The gastro rectal diagnostic transducer system of claim 8, wherein the body portion has a generally cylindrical shape, wherein the elongated openings are provided around a periphery of the body portion and wherein the base portion is outwardly flared proximate an end thereof that is opposite the body portion.

10. The gastro rectal diagnostic transducer system of claim 8, and further comprising a temperature sensor provided on at least one of the head portion and the body portion.

11. The gastro rectal oscopy diagnostic transducer of claim 8, wherein the head portion detachably engages the body portion and wherein the based portion detachably engages the body portion.

12. A method of using a gastro rectal oscopy system comprising:
providing a gastro rectal oscopy system comprising a body portion, a head portion and a base portion, wherein the body portion has a proximal end and a distal end, wherein intermediate the proximal end and the distal end, the body portion has a plurality of elongated openings formed therein, wherein the head portion is attached to the distal end, wherein the head portion has at least one light source mounted therein, wherein the base portion is attached to the proximal end, wherein the base portion has a control provided thereon and a connector that is operably attached to the inflatable device for changing the inflatable device between the uninflated configuration and the inflated configuration;
at least partially inserting the gastro rectal oscopy system into a rectum of a person;
inflating an inflatable device that is provided inside of the body portion from an uninflated configuration to an inflated configuration, wherein when the inflatable device is in the uninflated configuration, the inflatable device does not extend above an outer surface of the body portion and wherein when the inflatable device is in the inflated configuration, the inflatable device extends through the plurality of openings above the outer surface of the body portion; and
emitting vibrations using a vibratory device that is positioned inside of at least one of the body portion, the head portion and the base portion.

13. The method of using the gastro rectal oscopy system of claim 12, wherein the body portion has a generally cylindrical shape, wherein the elongated openings are provided around a periphery of the body portion and wherein the base portion is outwardly flared proximate an end thereof that is opposite the body portion.

14. The method of using the gastro rectal oscopy system of claim 12, wherein each of the elongated openings has a length and a width, wherein the elongated opening length is between about 5 times and about 10 times greater than the elongated opening width and wherein the elongated opening length is between about 50 percent and about 80 percent of a length of the body portion.

15. The method of using the gastro rectal oscopy system of claim 12, wherein the at least one light source comprises a first light source and a second light source and wherein the method further comprises:
emitting light having a first wavelength from the first light source; and
emitting light having a second wavelength from the second light source, wherein the first wavelength is different that the second wavelength.

16. The method of using the gastro rectal oscopy system of claim 15, wherein the first wavelength is about 660 nanometers and wherein the second wavelength is about 460 nanometers.

17. The method of using the gastro rectal oscopy system of claim 12, wherein the inflatable device is inflatable with air and wherein the head portion detachably engages the body portion and wherein the based portion detachably engages the body portion.

18. The method of using the gastro rectal oscopy system of claim 12, and further comprising:
engaging the base portion with an adapter handle; and
controlling at least one of light, sonic wave emission and camera using the adapter handle.

19. The method of using the gastro rectal oscopy system of claim 12, wherein contact of the inflatable device with tissue in the rectum causes sonic waves to be transferred to rectal nerves and muscles.

20. The method of using the gastro rectal oscopy system of claim 12, wherein the gastro rectal oscopy system increases blood flow in the rectum and reduces pain in the rectum.
